(19) United States
(12) Patent Application Publication
(10) Pub. No.: US 2011/0004273 A1
(43) Pub. Date: Jan. 6, 2011

(54) SOUND COMMAND TO STIMULATION CONVERTER

(75) Inventors: Koen Van den Heuvel, Hove (BE); Paul Carter, West Pennant Hills (AU)

Correspondence Address: KILPATRICK STOCKTON LLP, 1100 Peachtree Street, Suite 2800, ATLANTA, GA, 30309, US

(73) Assignee: COCHLEAR LIMITED, Lane Cove (AU)

(21) Appl. No.: 12/497,335

(22) Filed: Jul. 2, 2009

(51) Int. Cl. H04R 25/00; A61N 1/05

(52) U.S. Cl. 607/57

(57) ABSTRACT

A method and system for stimulating a tissue-stimulating prosthesis is disclosed. The method and system comprise receiving a sound command, such a MIDI command, and converting the sound to a stimulation signal. The stimulation signal is then used to provide stimulation to a recipient so that the recipient may perceive sound in accordance with the received sound command.

### BACKGROUND

1. Field of the Invention

The present invention relates generally to a tissue stimulating prosthesis, and more particularly to converting a sound command to electrical stimulation.

2. Related Art

A variety of implantable medical devices have been proposed to deliver controlled electrical stimulation to a region of a subject's body to perform a desired function. One such device which has been successful in providing hearing sensation to individuals with sensorineural hearing loss is the cochlear implant. For individuals with sensorineural hearing loss, there is typically damage to or an absence of hair cells within the cochlea which convert acoustic signals into nerve impulses which are perceived as sound by the brain. Such individuals are unable to derive suitable benefit from conventional hearing aid systems, and hence look to rely upon cochlear implants to provide them with the ability to perceive sound.

Cochlear implants use electrical stimulation of auditory nerve cells to bypass absent or defective hair cells that normally transduce acoustic vibrations into neural activity. Such devices generally use an array of electrode contacts implanted into the scala tympani of the cochlea so that the stimulation may differentially activate auditory neurons that normally encode differential frequencies of sound.

Auditory brain stimulators are used to treat a smaller number of recipients with bilateral degeneration of the auditory nerve. For such recipients, the auditory brain stimulator provides stimulation of the cochlear nucleus in the brainstem. Auditory brain stimulators similarly use a plurality of electrode contacts to provide stimulation to the recipient.

### SUMMARY

In one aspect of the present invention there is provided a method for providing stimulation via a stimulating lead assembly comprising a plurality of electrodes, the method comprising: receiving a sound command specifying a sound, wherein the sound command is in compliance with a musical instrument communication protocol; converting the sound command to at least one stimulation command specifying stimulation to be provided by one or more electrodes of the stimulating lead assembly; and providing electrical stimulation in response to the stimulation command via one or more of the plurality of electrodes.

In a second aspect of the invention, there is provided a system for providing stimulation comprising: a processor configured to receive a sound command specifying a sound, wherein the sound command is in compliance with a musical instrument communication protocol, and convert the sound command to at least one stimulation command specifying at least one stimulation signal to be provided by one or more electrodes of a stimulating lead assembly.

In a third aspect there is provided a system for providing stimulation, the method comprising: means for receiving a sound command specifying a sound, wherein the sound command is in compliance with a musical instrument communication protocol; means for converting the sound command to at least one stimulation command specifying stimulation to be provided by one or more electrodes of a stimulating lead assembly.

In a fourth aspect there is provided a method for providing stimulation via a stimulating lead assembly comprising a plurality of electrodes, the method comprising: receiving a sound command specifying a sound, wherein the sound command is in compliance with a musical instrument communication protocol; converting the sound command to at least one data signal specifying at least one stimulation signal to be provided by one or more electrodes of the stimulating lead assembly; and delivering the stimulation signal via one or more of the plurality of electrodes of the stimulating lead assembly.

In a fifth aspect there is provided a cochlear prosthesis comprising: a stimulating lead assembly comprising a plurality of electrode contacts; a sound processor configured to receive a sound command specifying a sound, wherein the sound command is in compliance with a musical instrument communication protocol, and convert the sound command to at least one data signal specifying at least one stimulation signal to be provided via one or more electrode contacts of the stimulating lead assembly; and a stimulator unit configured to receive the at least one data signal, generate the at least one stimulation signal, and provide the at least one stimulation signal to the stimulating lead assembly for providing the stimulation signal to a cochlea of a recipient via the one or more electrode contacts.

### BRIEF DESCRIPTION OF THE DRAWINGS

Embodiments of the present invention are described below with reference to the attached drawings, in which:

FIG. 1 is a perspective view of a cochlear implant in which embodiments of the present invention may be implemented;

FIG. 2 is a functional block diagram of the cochlear implant of FIG. 1, in accordance with an embodiment of the invention;

FIG. 3 illustrates a mapping of MIDI notes to an electrode contacts, in accordance with an embodiment of the invention;

FIG. 4 illustrates an exemplary computer that may be used for converting a sound command to a stimulation command, in accordance with an embodiment of the invention;

FIG. 5 provides a functional diagram of a sound processor, in accordance with an embodiment of the invention;

FIG. 6A is a simplified flow chart for converting a sound command to a stimulation signal, in accordance with an embodiment;

FIG. 6B is a more detailed flow chart for converting a sound command to a stimulation signal, in accordance with an embodiment;

FIG. 7 illustrates an alternative embodiment of a sound processor, in accordance with an embodiment of the invention; and

FIG. 8 illustrates yet another exemplary embodiment of a system for converting sound commands to stimulation signals, in accordance with an embodiment.

### DETAILED DESCRIPTION

Embodiments of the present invention are generally directed to converting a sound command, such as a Musical Instrument Digital Interface (MIDI) command, to stimulation. This stimulation may be applied by a tissue stimulating device, such as cochlear implant, so that a recipient of the tissue stimulating device may perceive sound in accordance with the sound command.

Embodiments of the present invention are described herein primarily in connection with one type of tissue stimulating device, a hearing prosthesis, namely a cochlear prosthesis (commonly referred to as cochlear prosthetic devices, cochlear implants, cochlear devices, and the like; simply “cochlea implants” herein.) Cochlear implants deliver electrical stimulation to the cochlea of a recipient. As used herein, cochlear implants also include hearing prostheses that deliver electrical stimulation in combination with other types of stimulation, such as acoustic or mechanical stimulation (sometimes referred to as mixed-mode devices). It would be appreciated that embodiments of the present invention may be implemented in any cochlear implant or other hearing prosthesis now known or later developed, including auditory brain stimulators, or implantable hearing prostheses that mechanically stimulate components of the recipient's middle or inner ear. For example, embodiments of the present invention may be implemented, for example, in a hearing prosthesis that provides mechanical stimulation to the middle ear and/or inner ear of a recipient.

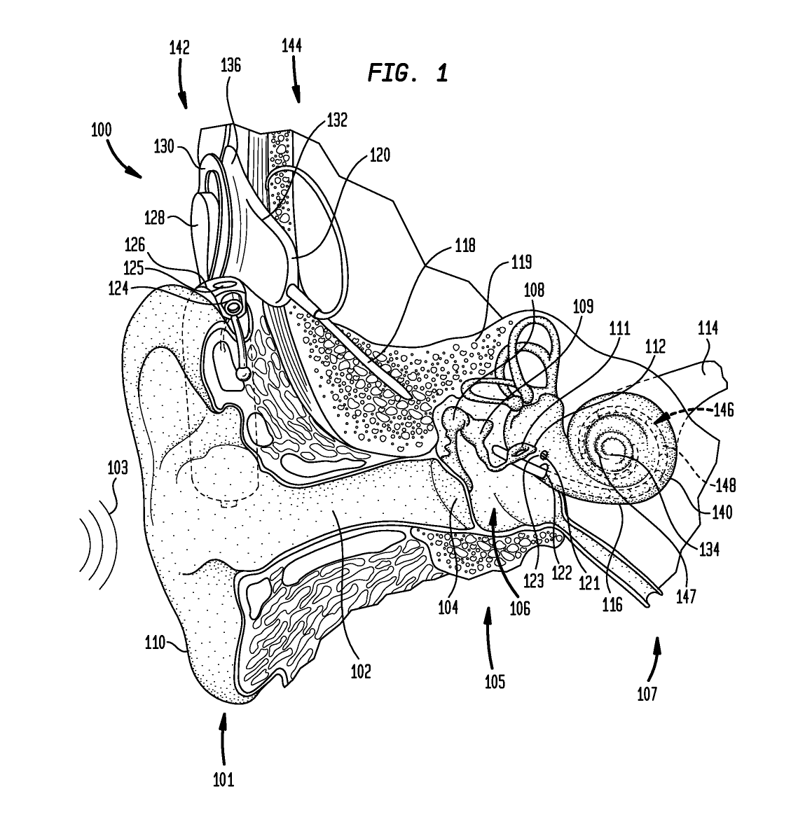

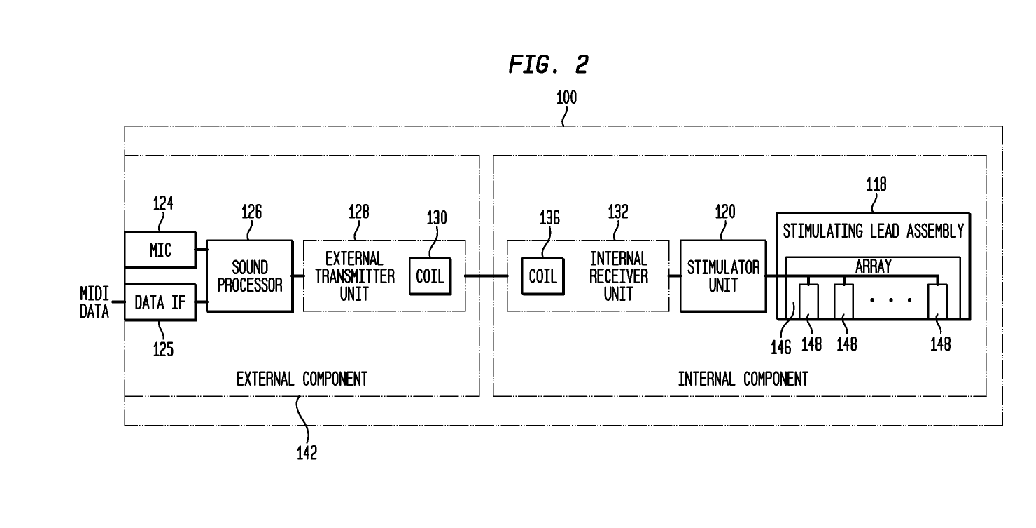

FIG. 1 is perspective view of a cochlear implant, referred to as cochlear implant 100 implanted in a recipient. FIG. 2 is a functional block diagram of cochlear implant 100. The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Cochlear implant 100 comprises an external component 142 which is directly or indirectly attached to the body of the recipient, and an internal component 144 which is temporarily or permanently implanted in the recipient. External component 142 typically comprises one or more sound input elements, such as microphone 124 for detecting sound, a sound processor 126, a power source (not shown), and an external transmitter unit 128. External transmitter unit 128 comprises an external coil 130 and, preferably, a magnet (not shown) secured directly or indirectly to external coil 130. Sound processor 126 processes the output of microphone 124 that is positioned, in the depicted embodiment, by auricle 110 of the recipient. Sound processor 126 generates encoded signals, sometimes referred to herein as encoded data signals, which are provided to external transmitter unit 128 via a cable (not shown). Sound processor 126 may further comprise a data input interface 125 that may be used to connect sound processor 126 to a data source, such as a personal computer or musical instrument (e.g., a MIDI instrument).

Internal component 144 comprises an internal receiver unit 132, a stimulator unit 120, and an electrode assembly 118. Internal receiver unit 132 comprises an internal coil 136, and preferably, a magnet (also not shown) fixed relative to the internal coil. Internal receiver unit 132 and stimulator unit 120 are hermetically sealed within a biocompatible housing, sometimes collectively referred to as a stimulator/receiver unit. The internal coil receives power and stimulation data from external coil 130. Electrode assembly 118 has a proximal end connected to stimulator unit 120, and a distal end implanted in cochlea 140. Electrode assembly 118 extends from stimulator unit 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrode contacts 148, sometimes referred to as array of electrode contacts 146 herein. Although array of electrode contacts 146 may be disposed on electrode assembly 118, in most practical applications, array of electrode contacts 146 is integrated into electrode assembly 118. As such, array of electrode contacts 146 is referred to herein as being disposed in electrode assembly 118. Stimulator unit 120 generates stimulation signals which are applied by electrode contacts 148 to cochlea 140, thereby stimulating auditory nerve 114. Because, in cochlear implant 100, electrode assembly 118 provides stimulation, electrode assembly 118 is sometimes referred to as a stimulating lead assembly.

In cochlear implant 100, external coil 130 transmits electrical signals (that is, power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding (not shown). In use, implantable receiver unit 132 may be positioned in a recess of the temporal bone adjacent auricle 110 of the recipient.

In an embodiment, a sound command is converted to electrical stimulation that is applied by stimulating lead assembly 118. This may enable a recipient of cochlear implant 100 to perceive sound in accordance with the sound command. The below described embodiments will primarily be described in the context of embodiments in which the sound commands are in compliance with one type of musical instrument communications protocol, the MIDI protocol. It should be noted, however, that although the present embodiment is discussed with reference to the MIDI protocol, in other embodiments, other musical instrument communications protocols may be used, such as the Open Sound Control (OSC) protocol developed at the Center for New Music and Audio Technologie (CNMAT), the mLan protocol developed by Yamaha, or the HD protocol presently being developed by the MMA.

The following provides a brief overview of the MIDI protocol. After which, exemplary embodiments will be described in which MIDI commands are converted to electrical stimulation applied using a cochlear implant, such as the above-described cochlear implant 100.

As would be appreciated by those of ordinary skill in the art, the Musical Instrument Digital Interface (MIDI) is an industry standard electronic communications protocol corresponding to a set of predetermined commands for generating sounds. The official MIDI standards are jointly developed and published by the MIDI Manufacturers Association (MMA) in Los Angeles, Calif., USA (see for example http://www.midi.org), and in Japan, the MIDI Committee of the Association of Musical Electronic Industry (AMEI) located in Tokyo (see for example http://www.amei.or.ip). The primary reference for the MIDI standard is The Complete MIDI 1.0 Detailed Specification, document version 96.1, which is available from MMA in English, or from AMEI in Japanese.

MIDI commands, also sometimes referred to a MIDI messages, are used in the MIDI protocol to specify sounds or a combination of sounds. For example, a MIDI command may define musical quantities such as pitch, frequency, loudness and other musical information relating to how to generate a sound.

MIDI commands may be generated by a musical instrument such as a synthesizer or, for example, by a MIDI software application. An example of one such MIDI software application is Rosegarden, which is a combined audio and MIDI sequencer, score editor, and general-purpose music composition and editing environment (see for example www.rosegardenmusic.com). A user may use MIDI software applications, such as Rosegarden, to create a musical composition comprising a plurality of MIDI commands. The MIDI commands may specify sounds corresponding to many instruments, each individually configured, and be organized such that when these plurality of sounds are combined, they create an orchestral effect.

A musical composition comprising a series of MIDI commands can be stored as a data file and loaded by a musical instrument or software capable of interpreting these commands. Alternatively a series of MIDI commands may be directly streamed to a musical instrument or software capable of interpreting the stream of commands in real time.

In an embodiment, a musical composition may be presented to a recipient of a cochlear implant by converting the MIDI commands for the musical composition to electrical stimulation signals. The stimulation signals may then be used to provide electrical stimulation to cochlea 140 via electrode contacts 148 of stimulating lead assembly 118.

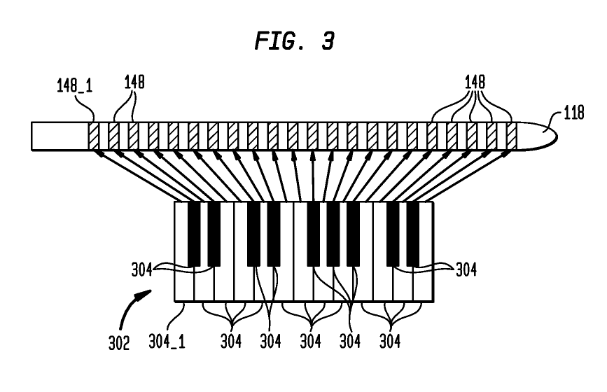

FIG. 3 illustrates one simple example of how a MIDI command might be converted to an electrical stimulation signal. As illustrated, each note 304 of a MIDI keyboard 302 may be mapped to a corresponding electrode contact 148 of stimulating lead assembly 118. Using such a map, a MIDI command specifying a particular note (i.e., key 304) and loudness may be converted to a stimulation signal for stimulating the corresponding electrode at a particular current level. For example, a MIDI command specifying that note 304_1 is to be presented to the recipient with a particular intensity may be converted to stimulation signal for stimulating electrode contact 148_1 at a particular current level.

The intensity (i.e., loudness) may be converted to a current level such that an MIDI intensity of 0 results in stimulation applied at the Threshold level (T-level) for electrode contact 148_1. A MIDI intensity of 127 may be converted to stimulation applied at the Comfort level (C-level) for electrode contact 148_1. The following formula may be used for converting the MIDI intensity level to a current level:

CL=(I/127)(C−T)+T,

where I is the MIDI intensity level, C is the C-level for the electrode contact, T is the T-level for the electrode contact, and the maximum MIDI intensity level is 127. It should be noted that this is but one exemplary mechanism for converting MIDI intensity levels to current levels, and other mechanisms may be used in other embodiments.

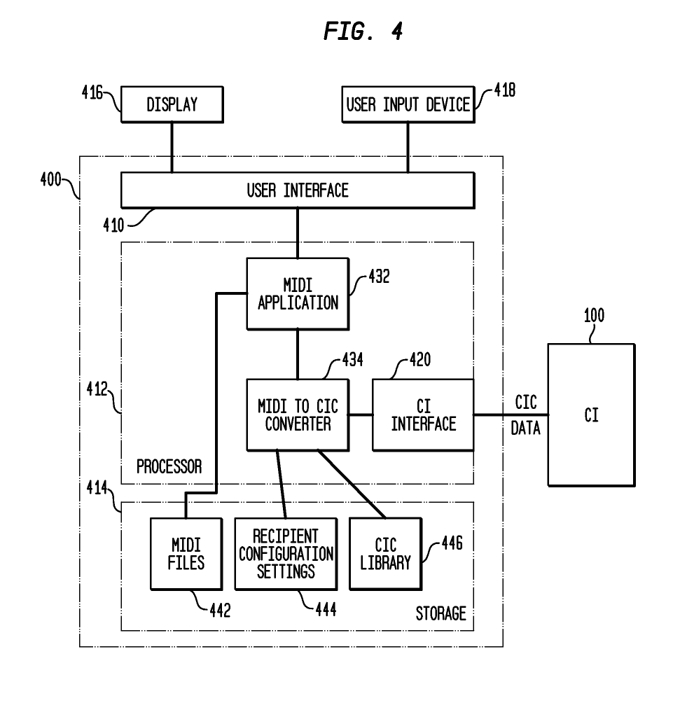

FIG. 4 illustrates an exemplary computer 400 that may be used for converting a sound command, such as MIDI command to a stimulation command. Computer 400 may be, for example, a commercially available computer comprising a user interface 410, a processor 412, a storage 414, and a CI interface 420. User interface 410 may connect computer 400 to one or more devices, such as a display 416 and one or more user input devices 418. Display 416 may be, for example, any type of display device, such as, for example, those commonly used with computer systems. User input devices 418 may be any type of interface capable of receiving information from a recipient, such as, for example, a computer keyboard, mouse, voice-responsive software, touch-screen (e.g., integrated with display 222), retinal control, joystick, and any other data entry or data presentation formats now or later developed.

Processor 412 may be any type of device or device(s) capable of executing instructions such as, for example, one or more microprocessors, digital electronic circuitry, integrated circuitry, specially designed ASICs (application specific integrated circuits), firmware, software, and/or combinations thereof. Storage 412 may comprise, for example, volatile and/or non-volatile storage, such as, Random Access Memory (RAM), a hard drive, etc.

CI interface 420 may be configured to connect computer 400 to cochlear implant 100 via for example, a cable or wireless connection. Any suitable type interface may be used for connecting CI interface 420, such as for example, a Universal Serial Bus (USB) interface, Bluetooth, etc. It should be understood that FIG. 4 is a simplified illustration of computer 400, and is provided to illustrate one exemplary system that may be used for converting a sound command to a stimulation command, such as a MIDI command. As used herein, the term stimulation command refers to a command specifying stimulation.

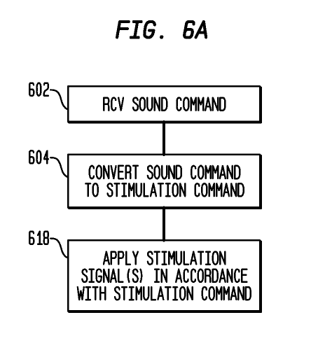

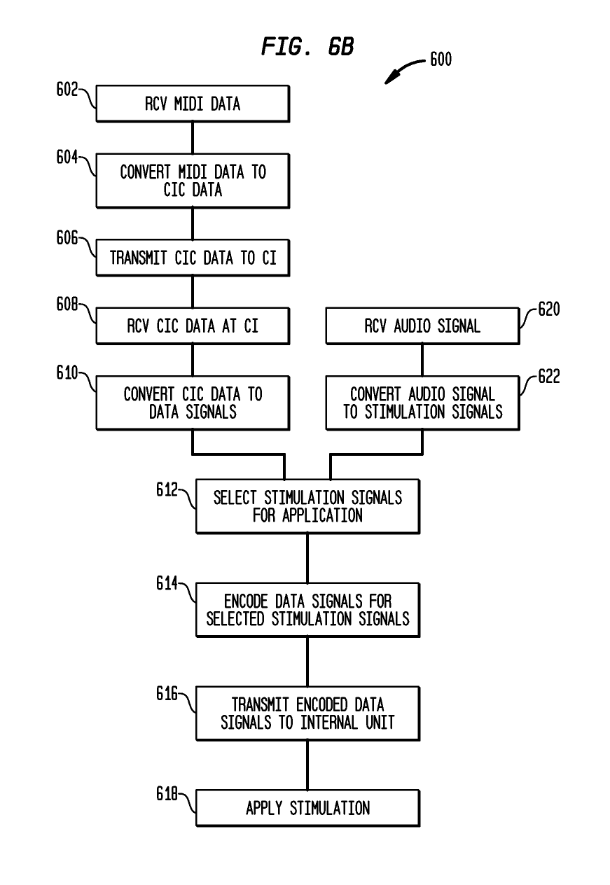

As illustrated, processor 412 may execute a MIDI application 432 as well as a MIDI to Cochlear Implant Communicator (CIC) conversion module 434. MIDI application 432 may be, for example, a commercially available MIDI application, such as the above noted Rosegarden application. CIC conversion module 434 may be software configured to take as an input a MIDI command (e.g., from MIDI application 432) and convert the command to a stimulation command. In an embodiment, CIC conversion module 434 may be configured in a similar manner to the Nucleus Implant Communicator (NIC) discussed in U.S. patent application Ser. No. 10/250,880, which is hereby incorporated by reference in its entirety. A further description of the exemplary operation of CIC conversion module will be presented below with reference to FIG. 6A-B.

Storage 412 may, for example, store one or more MIDI files 442, configuration settings 444 for the recipient of cochlear implant 100, and a CIC library 446. The MIDI files 442 may be standard MIDI files, such as for example MIDI files for presenting a musical composition to the recipient. Configuration settings 444 may comprise settings for the recipient's cochlear implant 100, such as, for example, the T-level and C-levels for each electrode contact 148 of stimulating lead assembly 118. CIC library 446 may store, for example, a mapping of MIDI command types to stimulation commands.

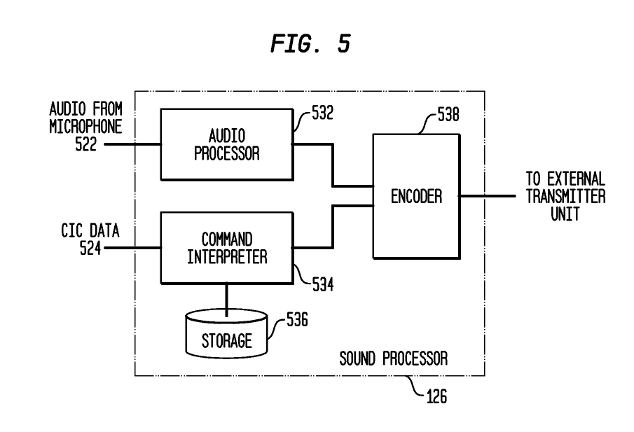

FIG. 5 provides a functional diagram of sound processor 126 (FIG. 1), in accordance with an embodiment of the invention. As illustrated, sound processor 126 receives audio input 522 from one or more sound input devices 124, such as microphone. It should be appreciated, however, that any sound input device now or later developed may be used to provide one or more input sound signals. For example, in an embodiment, the sound input device may be, for example, an input jack for receiving a signal from, for example, the headphone jack of an MP3 player or other audio device.

Additionally, sound processor 126 may receive data input 524 via data interface 125 (FIG. 1), which may be connected to CI interface 420 of computer 400 (FIG. 4). Sound processor 126 may comprise an audio processor 532 as well as a command interpreter 534. Audio processor 532 may be configured to convert received audio to data signals, and may function in manner similar to sound processors in presently available cochlear implants. For example, audio processor 532 may comprise a pre-processor, a filter bank, a maxima selector, etc. The operation of converting a received audio signal to a data signal is considered well known in the art, and as such is not described further herein.

Command interpreter 534 may be configured to convert stimulation commands, such as stimulation commands generated by MIDI-to-CIC converter 434 of computer 400 to data signals. A further description of an exemplary mechanism for converting stimulation commands to data signals is presented below with reference to FIG. 6A-B.

Encoder 538 may then encode the data signals from audio processor 532 and command interpreter 534. Encoder 538 may also arbitrate between data signals received from audio processor 532 and command interpreter 534, such as, for example, if a conflict arises.

Encoder 538 may then provide the encoded signals to external transmitter unit 128 (FIG. 1) for transmission to internal component 144 (FIG. 1). There are several speech coding strategies that may be used when converting sound into all electrical stimulation signals, such as, for example, Continuous Interleaved Sampling (CIS), Spectral PEAK Extraction (SPEAK), Advanced Combination Encoders (ACE), Simultaneous Analog Stimulation (SAS), MPS, Paired Pulsatile Sampler (PPS), Quadruple Pulsatile Sampler (QPS), Hybrid Analog Pulsatile (HAPs), n-of-m and HiRes™, developed by Advanced Bionics.

Internal receiver unit 132 then receives the encoded signals and provides the encoded signals to stimulator unit 120. Stimulator unit 120 then generates stimulation signals which are applied by electrode contacts 148 to cochlea 140, thereby stimulating auditory nerve 114.

FIG. 6A is a simplified flow chart for converting a sound command, such as a MIDI command, to a stimulation signal, in accordance with an embodiment. FIG. 6B illustrates a more detailed version of the flow chart of FIG. 6A. FIG. 6A and FIG. 6B will be described with reference to the above-discussed FIGS. 3-5. Further, for ease of explanation, the sound command will be discussed with reference to a MIDI command. It should, however, be understood that in other embodiments other type of sound commands may be converted to stimulation signals, such as, for example, an OSC command.

For ease of explanation, the more simplified FIG. 6A will first be described followed by the more detailed FIG. 6B. As illustrated in FIG. 6A, a sound command is first received at block 602 by MIDI-to-CIC converter 434. As noted above, the sound command may be in compliance with a musical instrument communications protocol, such as, MIDI, ORD, mLan, etc. MIDI-to-CIC converter 434 converts, at block 604, the sound command to a stimulation command specifying stimulation to be applied by one or more electrode contacts 148 of stimulating lead assembly 118. After which, the stimulation signals are applied via electrode contacts 148 of stimulating lead assembly 118 at block 618.

At block 602, a MIDI command is received by MIDI-to-CIC converter 434. This MIDI command may be received from MIDI Application 432. Additionally, as noted above, the MIDI command may be MIDI command for a MIDI file retrieved from a library of stored MIDI files 442. Or, for example, the MIDI command may be received in real time from a MIDI musical instrument, or generated in real time by a MIDI application 432.

For ease of explanation, the presently described embodiment will be described with reference to a MIDI command specifying that a note of 22 note MIDI keyboard, such as keyboard 302 (FIG. 3). Further, in this embodiment, each note 304 of keyboard 302 is mapped to a corresponding electrode contact 148 of stimulating lead assembly 118, such as described above with reference to FIG. 3. Further, in this example, the received MIDI command will specify the frequency of the note as well as the intensity (loudness) of the note.

MIDI-to-CIC converter 434 converts the received MIDI command to a stimulation command at block 604. In converting the MIDI command to a stimulation command, MIDI-to-CIC converter 434 may consult CIC library 446 to determine the type of sound command to be used. For example, MIDI-to-CIC converter 434 may analyze the received MIDI commands to determine the instrument(s) represented by the MIDI commands (i.e., the type of sound to be presented to the user). For simplicity, in this example, the MIDI commands are assumed to provide information for presenting piano keyboard sounds to the recipient.

MIDI-to-CIC converter 434 may analyze the received MIDI commands and access CIC library 446 to determine the type of stimulation command(s) to be used. As noted above, CIC library 446 may store information regarding a mapping between stimulation commands and types of MIDI sounds (e.g., keyboard, horn, etc.) In this example, it is assumed that a command entitled “STIMULATION(channel, level)” corresponds to the keyboard type of instrument, where “channel” specifies the channel (electrode contact 148) to be stimulated and “level” specifies the current level at which to stimulate the specified channel. MIDI-to-CI converter 434 may thus analyze the MIDI command(s) and determine that MIDI command contains information regarding notes 304 for the 22 note keyboard 302 (FIG. 3). MIDI-to-CI converter 343 may then access CIC library 446 to look up the stimulation command corresponding to this 22 note keyboard sound type, which in this example is “STIMULATION(channel, level).”

CIC library 446 may further store a mapping of notes 304 to electrode contacts 148 such as discussed above with reference to FIG. 3. Additionally, CIC library 446 may store a mapping for converting MIDI intensities to current levels, such as the above discussed formula: CL=(I/127)(C−T)+T.

MIDI-to-CIC converter 434 may access the mappings stored by CIC library 446 to determine the channel and level parameters for the STIMULATION command. Additionally, MIDI-to-CIC converter 434 may access the stored recipient configuration settings 444 in order to determine, for example, the recipient's T-level and C-levels for the channel to be stimulated. MIDI-to-CIC converter 434 may then compute the current level parameter using the above discussed formula, CL=(I/127)(C−T)+T.

It should be noted that this is but one example of an exemplary stimulation command type, and in other embodiments, different types of stimulation command(s) may be used. For example, if a horn type sound is to be presented to the user, the MIDI-to-CIC converter 434 may access the CIC library 446 to retrieve a stimulation command, such as HORNSTIMULATE(channel, level, duration), where channel specifies the center electrode for the sound, level specifies the intensity of the sound, and duration specifies the duration of the note to be presented. Or, for example, MIDI-to-CIC converter 434 may access the CIC library to retrieve a sequence of stimulation commands to be used for a particular type of sound. As one such example, a helicopter type sound may be presented to the recipient by MIDI-to-CIC converter 434 generating a repeating pattern of stimulation commands. It should also be noted that these stimulation commands are exemplary only and provided for describing one possible exemplary embodiment.

After converting the MIDI command to a stimulation command, MIDI-to-CIC converter 434 may transmit the stimulation command to cochlear implant 100 via CI interface 420 at block 606. The stimulation command may then be received by data interface 125 and transferred to sound processor 126 at block 608.

The command interpreter 534 of sound processor 126 converts the received sound commands to data signals for applying stimulation corresponding to the received sound command at block 610. In converting the sound command, command interpreter 534 may access the storage 536 to obtain information regarding a mapping between the stimulation command and the corresponding stimulation signal to be applied. For example, for the STIMULATE(channel,level) command, the command interpreter 534 may generate a data signal specifying a stimulation signal for stimulating the specified electrode at the specified current level.

Command interpreter 534 may obtain from storage 536 additional parameters for application of stimulation in response to the received STIMULATE command, such as the number and shape of pulses to be applied, the rate of application, and duration of the pulses to be applied. In other words, the specifics of the stimulation to be applied may vary depending on the type of stimulation command. For example, the HORNSTIMULATE(channel, level, duration) may be mapped by command interpreter 534 such that stimulation is applied on a plurality of electrode contacts 148 each with specific parameters so that the hearing sensation perceived by the recipient is different than the hearing sensation perceived by the recipient for the STIMULATE command.

Additionally, storage 536 may store recipient specific information, such as the number of maxima for cochlear implant, and/or an electrode shift to be implemented for the recipient. For example, stimulating lead assemblies 118 may be inserted differently for different patients. Thus, one patient may have deep insertion while another patient has a shallow insertion. As such, the frequency perceived by the recipient may depend on this depth of the insertion. In an embodiment, storage 536 may store an electrode shift to be applied that may help compensate for variances in the depth in which the recipient's cochlear implant is inserted.

Storage 536 may also store the maximum number of stimulation signals (number of maxima) that the recipient's cochlear implant may simultaneously apply. For example, if the musical composition is such that a 3 note chord is to be presented to a recipient whose cochlear implant is configured to only apply 2 maxima, storage 536 may store information that enables 534 to select which 2 notes to be presented to the recipient. As an example, a particular recipient may perceive higher frequencies better than lower frequencies. Thus, for such a recipient, storage 536 may store information such that command interpreter 534 selects the two highest frequencies in the event multiple notes are to be simultaneously presented to such a recipient. It should be noted that this is but one simple example provided to demonstrate how the specifics of the stimulation signals generated by cochlear implant 100 may vary by recipient.

Sound processor 126 may also receive audio signals 522 from microphone 124 (or other audio source), at block 622, simultaneous with receipt of the sound commands 524. Audio processor 532 may then generate data signals representative of the received audio signals 522 at block 622. As noted above, generation of data signals from audio signals is well known to those of skill in the art, and, as such is not described further herein. In another embodiment, sound processor 126 may comprise a user interface that permits the recipient to turn off the microphone 124 when the recipient desires to listen to music, such that sound processor 126 does not generate data signals for audio signals 522 but only for sound commands 524.

Encoder 538 receives the data signals from command interpreter 534 and audio processor 532 and may, for example, select which of the stimulation signals specified by the received data signals are to be applied. For example, if 2 MIDI stimulation signals are received and 2 audio stimulation signals are received and cochlear implant 100 is configured to apply 3 maxima, encoder 538 may select the maxima to be applied. Encoder 538 may use various strategies for selecting amongst a plurality of stimulation signals. Moreover, the strategy implemented may be recipient and/or cochlear implant specific.

Encoder 538 may then encode the data signals at block 614 for transmission to internal component 144 via external transmitter unit 128. The encoded signals may then be provided to external transmitter unit 128 and transmitted to the internal component 144 at block 616. At block 618, stimulator unit 120 may generate stimulation signals that are applied by stimulating lead assembly 118 based on the received data signals.

It should be understood that although the above discussed flow chart 600 was discussed with regard to mapping a single sound command to a single stimulation signal, it should be understood that in implementations, a plurality of sound commands could be mapped to a plurality of stimulations signals, one sound command might be mapped to a plurality of stimulation signals, or a plurality of stimulation signals might be mapped to a single stimulation signal.

Further, it should be understood that the above-discussed embodiments were discussed with reference to a cochlear implant that provides electrical stimulation. It should be understood, however, that embodiments of the present invention may also be implemented in tissue stimulating devices that provide other types of stimulation, such as optical stimulation or mechanical stimulation to the recipient's cochlea. Optical stimulation system may use a stimulating lead assembly comprising optical contacts for delivering optical stimulation. A further description of an exemplary tissue stimulating device providing optical stimulation is provided in U.S. patent application Ser. No. 12/348,225, filed Jan. 2, 2009 and entitled “Combined Optical and Electrical Neural Stimulation,” which is hereby incorporated by reference. In a cochlear implant providing a mechanical stimulation to the middle ear or inner ear of the recipient's cochlea, a stimulation signal may generated by a stimulator unit provided to a transducer that generates mechanical movement. A rod or other device then transmits this mechanical movement to a component of the recipient's middle or inner ear (e.g., the stapes, oval window, etc.). A further description of exemplary cochlear implants providing mechanical stimulation is provided in U.S. Pat. No. 5,277,694, U.S. Pat. No. 6,123,660, U.S. Pat. No. 6,162,169, and the International Application No.: PCT/US09/38932, entitled “Objective Fitting of a Hearing Prosthesis,” filed Mar. 31, 2009 (Attorney Docket: 22409-00501), each of which are hereby incorporated by reference.

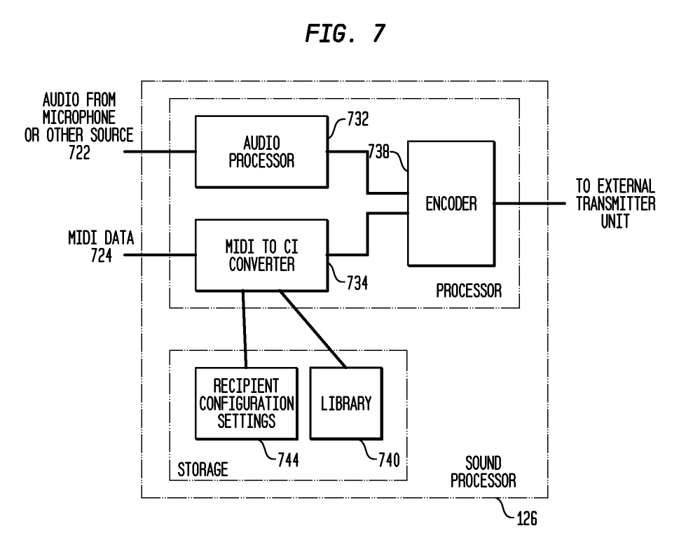

FIG. 7 illustrates an alternative embodiment in which sound processor 126 comprises a MIDI-to-CI converter 702. As illustrated, sound processor 126 may receive MIDI command 734. MIDI-to-CI converter 734 may then convert the received MIDI command to data signals specifying the application of stimulation signals via stimulating lead assembly 118. Because in this embodiment the MIDI command is converted to data signals specifying the stimulation signals within sound processor 126, the MIDI command is not first converted to a stimulation command, but instead converted directly to the data signals specifying the stimulation signals.

In converting MIDI 724 data to the data signals, MIDI-to-CI converter 734 may access a storage 740 comprising data regarding the recipient's configuration settings 744 and a library 746. The recipient configurations settings 744 may comprise T-levels and C-levels for cochlear implant 100.

MIDI-to-CI converter 734 may analyze the MIDI command 724 to determine the type of sound (e.g., piano, horn, strings, etc.) to be presented to the recipient. MIDI-to-CI converter 734 may then access library 746, which stores information mapping sound types to corresponding stimulation signal types, and determine the type and specifics of the stimulation signal(s) to be applied. The information stored by library 746 may comprise, for example, stimulation parameters for the stimulation signal type, such as information specifying the number and orientation of electrodes to be stimulated, as well information specifying the pulses to be applied on the electrodes, such as the number of pulses to be applied, the pulse rate, pulse duration, pulse shape, etc.

As an example, referring back to FIG. 3, if the MIDI command specifies that a particular piano note 304_1 is to be played, the MIDI-to-CI converter 734 may access library 746, which maps the piano note type to a stimulation signal type specifying that a single electrode is to be stimulated at a particular current level. Additionally, MIDI-to-CI converter 734 may retrieve information from library 746 regarding the pulse parameters for applying this stimulation.

In addition to determining the type of stimulation signal, MIDI-to-CI converter 734 may further calculate the current level for applying the stimulation. For example, MIDI-to-CI converter 734 may access the stored recipient configuration settings 744 to obtain the parameters and formula for calculating this current level. As noted above, in one example, the current level may be calculated using a formula, such as, CL=(I/127)(C−T)+T, where T is the T-level and C is the C-level for the electrode. Using this information, MIDI-to-CI converter 734 may, in this embodiment, generate data signals specifying the stimulation signal(s) to be applied for the received MIDI command.

After generating data signals specifying the stimulation signal(s), MIDI-to-CI converter 734 may send the data signals to encoder 738 that then forwards the encoded signals to the internal component 144 of the cochlear implant 100 for application of the specified stimulations signals by stimulating lead assembly 118. Encoder 738 and audio processor 732 may function in a similar manner to encoder 538 and audio processor 532 of the above discussed FIG. 5.

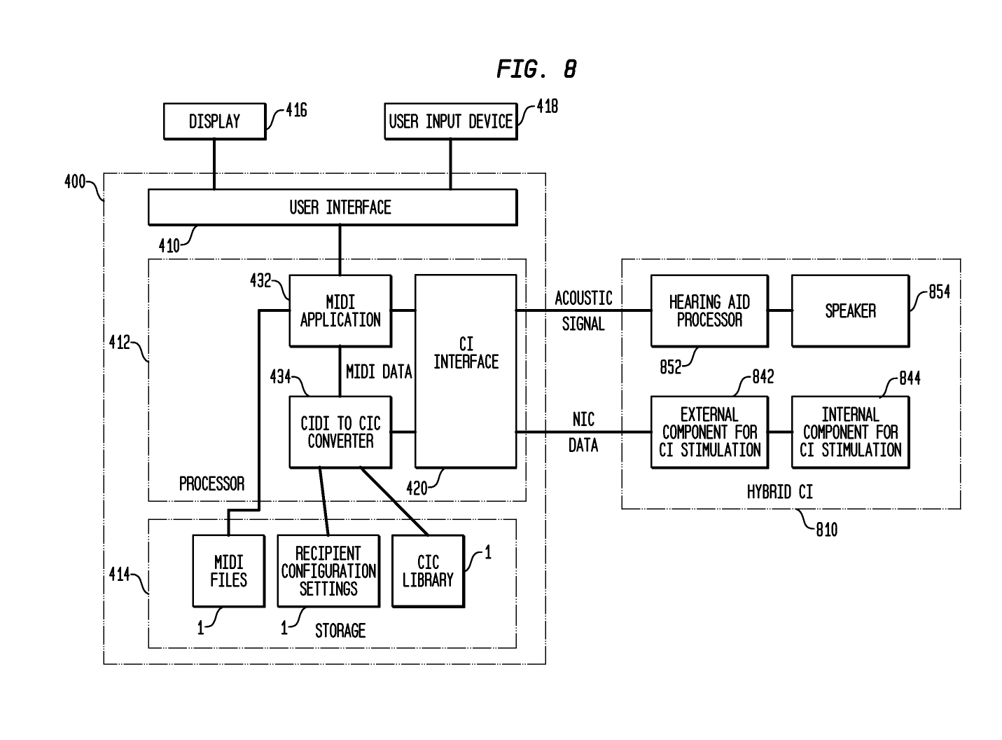

FIG. 8 illustrates yet another exemplary embodiment of a system for converting sound commands to stimulation signals, in accordance with an embodiment. In this embodiment, computer 400 may be identical to computer 400 of FIG. 4, and MIDI application 432 is capable of outputting both MIDI command and an acoustic signal comprising an acoustic version of the song. This acoustic version may be suitable for playing over a speaker or headphones. MIDI application 432 may be for example, a commercially available MIDI software package.

MIDI-to-CIC converter 434 may convert the MIDI commands from application 432 to sound commands (referred to as CIC data in this example), such as was described above with reference to FIGS. 4-6. The CIC Data as well as the acoustic version of the sound may then be provided to a hybrid cochlear implant system 810. This acoustic version may be provided to the hybrid cochlear implant 810 via, for example, a wire connecting computer 400 and hybrid cochlear implant 810. Or, for example, the acoustic version may be played over a speaker (e.g., headphones) and received by a microphone of hybrid cochlear implant 810.

As is known to those of skill in the art, a hybrid cochlear implant is capable of providing both electrical stimulation as well as acoustic stimulation. The electrical stimulation may be applied using, for example, a stimulating lead assembly, such as stimulating lead assembly 118 of the above-described FIG. 1. The acoustic stimulation may be provided in a manner similar to a hearing aid, such as, for example, using a speaker.

Hybrid cochlear implants may be helpful for recipients who have lost hearing for higher frequencies, but can still hear lower frequencies with the help of a hearing aid. Thus, for such recipients, electrical stimulation may be applied for higher frequencies using, for example, a short stimulating lead assembly positioned partially within the outer portion of cochlea 140; and, lower frequencies may be provided using the hearing aid portion of the hybrid cochlear implant. A further description of one type of hybrid cochlear implant is provided in Gantz B J, Turner C., “Combining Acoustic and Electrical Speech Processing: Iowa/Nucleus Hybrid Implant,” Acta Otolaryngol 304; 124(4): 344-7, which is hereby incorporated by reference.

As illustrated, hybrid cochlear implant 810 may comprise an external component 842 and an internal component 844. External component 842 and internal component 844 may function in a similar manner to external component 142 and internal component 144 of the above-discussed FIGS. 1-6. For example, external component 142 may comprise sound processor 126 including a command interpreter 534 (FIG. 5) for converting the sound commands received from computer 400 to data signals specifying the stimulation signals to be applied.

Hybrid cochlear implant 810 may further include a hearing aid portion comprising a hearing aid processor 852 and a speaker 856. Although hearing aid processor 852 is illustrated as separate from external component 810, it should be understood that the figure was illustrated in this manner to show the acoustic and electrical stimulation paths. And, in actual implementation, hearing aid processor 852 may be included, for example, in the sound processor 126.

In operation, the sound commands from MIDI-to-CIC converter 434 are provided to the external component 842 where they are converted by the command interpreter 534 (FIG. 5) to data signals specifying the stimulation signals to be applied via electrode contacts 148 of stimulating lead assembly 118. Additionally, the command interpreter 534 in this example may be configured so that command interpreter 534 only specifies stimulation signals within a particular frequency band. For example, as noted above, in certain implementations, electrical stimulation is only provided for high frequencies. Storage 536 may, for example, store the cut-off frequency, below which electrical stimulation is not applied. Command interpreter 534 may retrieve and use this cut-off frequency so that command interpreter 534 only specifies the generation of stimulation signals for frequencies above this cut-off frequency.

Parallel to processing the sound commands, the acoustic version is provided to the hearing aid processor 852 that processes and delivers the acoustic version of the received sound using speaker 854. Thus, in this embodiment, hybrid cochlear implant 810 can deliver lower frequency sounds via the hearing aid portion; and, higher frequency sounds via electrical stimulation. In embodiments, hearing aid processor 852 may optionally include a low pass filter that filters out frequencies below the cut-off frequency.

It should be noted that although the embodiments of FIGS. 7 and 8 were discussed with reference to providing electrical stimulation, in other embodiments, other types of stimulation may be applied, such as optical, mechanical, or a combination of optical, mechanical, and/or electrical stimulation may be used.

As would be appreciated by those skilled in the art, the sequence of MIDI commands or cochlear implant (CI) music that corresponds to a music piece for a cochlear implant recipient may have a number of differences when compared to the sequence of MIDI commands corresponding to the same piece music that would be applicable to a standard MIDI playing device intended for an audience having “normal” perception of sound. This may be due to the CI music having to take into account the performance characteristics of a cochlear implant when reproducing or playing a sequence of MIDI commands. As such, CI music may be specifically customized or tailored for cochlear implant recipients.

This customization or tailoring process may allow a large degree of scope for artistic input in the generation of MIDI commands that a cochlear implant recipient will find aesthetically pleasing. As with all artistic endeavors, there may be those persons who will be particularly skilled in this process of composing CI music. Because CI music may be stored as MIDI file, CI music may be readily exchangeable by implant recipients, in a similar manner to the exchange of music files in the MP3 format. As an example, CI music files may be uploaded to a central site where other implant recipients may be able to download them for a fee to be uploaded to stimulate their cochlear implants.

In another embodiment, an intermediate file format is provided for storing CI music that involves storing the sequence of stimulation commands, such as those discussed above as generated by MIDI-to-CI converter 434. These storage commands may include a free parameter for recipient dependent configurations settings such as T-level and C-level. On loading of this intermediate file format in a computer, software may populate storage commands with the recipient dependent settings and then played.

In yet another embodiment, a raw file format is provided that corresponds to the sequence of electrode stimulations and the associated parameters such as current level, pulse width and rate that corresponds with the MIDI commands that have been converted to a stimulation pattern. This raw file format may be loaded directly by the cochlear implant via the sound processor. As would be appreciated by those skilled in the art, this raw file format will generally correspond to a particular recipient as it will be based on their cochlear implant configuration settings.

All documents, patents, journal articles and other materials cited in the present application are hereby incorporated by reference.

Embodiments of the present invention have been described with reference to several aspects of the present invention. It would be appreciated that embodiments described in the context of one aspect may be used in other aspects without departing from the scope of the present invention.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. A method for providing stimulation to a cochlea of a recipient, the method comprising:
receiving a sound command specifying a sound, wherein the sound command is in compliance with a musical instrument communication protocol;
converting the sound command to at least one stimulation command specifying stimulation to be provided to the cochlea; and
providing stimulation to the cochlea in accordance with the stimulation command.

2. The method of claim 1, wherein providing stimulation to the cochlea comprises:
providing electrical stimulation in response to the stimulation command via one or more of a plurality of electrodes of a stimulating lead assembly.

3. The method of claim 1, wherein providing electrical stimulation comprises:
generating at least one stimulation signal in accordance with the stimulation command; and
delivering the stimulation signal to a cochlea of a recipient of the stimulating lead assembly via one or more of the plurality of electrodes.

4. The method of claim 1, wherein converting the sound command to a stimulation command comprises:
obtaining at least one stimulation command corresponding to the sound command from a library of stored stimulation commands.

5. The method of claim 4, wherein obtaining at least one stimulation command comprises:
obtaining a sequence of a plurality of stimulation commands from the library, wherein the sequence specifies stimulation to be provided in accordance with the received sound command.

6. The method of claim 4, wherein converting the sound command to a stimulation command further comprises:
obtaining at least one recipient parameter from a set of one of or more stored recipient parameters for the recipient;
specifying at least one parameter of the of the stimulation command in accordance with the obtained at least one recipient parameter.

7. The method of claim 6, wherein the obtained at least one recipient parameter comprises at least one of a threshold level, a comfort level, a pulse width, and a stimulation rate stored for the recipient.

8. The method of claim 1, wherein the received sound command is a Musical Instrument Digital Interface (MIDI) command.

9. The method of claim 8, wherein the received MIDI command is a MIDI command for a musical work comprising a plurality of MIDI commands for presentation to the recipient.

10. The method of claim 9, wherein the plurality of MIDI commands are stored in a file.

11. The method of claim 1, further comprising:
receiving an audio signal;
generating a signal representative of the received audio signal; and
delivering the signal representative of the received audio signal to the recipient.

12. The method of claim 11, wherein delivering the signal representative of the received audio signal comprises:
providing the signal representative of the received audio signal to a speaker configured to generate sound based on the provided signal.

13. The method of claim 1, wherein providing stimulation to the cochlea comprises:
providing mechanical stimulation in response to the stimulation command to at least one of an inner ear and an outer ear of the recipient.

14. A system for providing stimulation comprising:
a processor configured to receive a sound command specifying a sound, wherein the sound command is in compliance with a musical instrument communication protocol, and convert the sound command to at least one stimulation command specifying at least one stimulation signal to be provided to a cochlea of a recipient.

15. The system of claim 14, further comprising:
a storage storing a library of stimulation commands; and
wherein the processor in converting the sound command to a stimulation command is further configured to obtain at least one stimulation command corresponding to the sound command from the library of stored stimulation commands.

16. The system of claim 15, wherein the processor in obtaining at least one stimulation command is further configured to obtain a sequence of a plurality of stimulation commands from the library, wherein the sequence specifies stimulation to be provided in accordance with the received sound command.

17. The system of claim 16, wherein the processor in converting the sound command to a stimulation command is further configured to obtain at least one recipient parameter from a set of one of or more stored recipient parameters for the recipient, and specify at least one parameter of the of the stimulation command in accordance with the obtained at least one recipient parameter.

18. The system of claim 17, wherein the at least one recipient parameter comprises at least one of a threshold level, a comfort level, a pulse width, and a stimulation rate stored for the recipient.

19. The system of claim 14, wherein the sound command is a Musical Instrument Digital Interface (MIDI) command.

20. The system of claim 19, wherein the received MIDI command is a MIDI command for a musical work comprising a plurality of MIDI commands for presentation to the recipient.

21. The system of claim 20, wherein the plurality of MIDI commands are stored in a file.

22. The system of claim 13, further comprising:
a cochlear prosthesis comprising:
a processor configured to receive the stimulation command, and convert the stimulation command to a data signal specifying a stimulation signal; and
a stimulating lead assembly comprising one or more electrode contacts configured to deliver the stimulation signal using one or more of the electrode contacts.

23. The system of claim 22, wherein the processor is further configured to provide an audio signal and wherein the cochlear prosthesis further comprises:
a speaker; and
wherein the processor comprises an audio processor configured to receive the audio signal, generate a signal representative of the received audio signal, and provide the signal representative of the audio signal to the speaker for generating audio.

24. The system of claim 22, wherein the processor of the cochlear prosthesis is further configured to receive an audio signal, and generate a data signal specifying a stimulation signal representative of the received audio signal.

25. The system of claim 24, wherein the cochlear prosthesis further comprises a microphone for receiving audio and generating the audio signal.

26. The system of claim 14, further comprising:
a stimulation arrangement configured to mechanically stimulate at least one of an inner ear or a middle ear of the recipient in response to the stimulation command.

27. A system for providing stimulation, the method comprising:
means for receiving a sound command specifying a sound, wherein the sound command is in compliance with a musical instrument communication protocol;
means for converting the sound command to at least one stimulation command specifying stimulation to be provided to a cochlea of a recipient.

28. The system of claim 27, wherein the received sound command is a Musical Instrument Digital Interface (MIDI) command.

29. The system of claim 28, wherein the received MIDI command is a MIDI command for a musical work comprising a plurality of MIDI commands for presentation to the recipient.

30. The system of claim 27, further comprising:
means for generating at least one stimulation signal in accordance with the stimulation command; and
means for delivering the stimulation signal to a cochlea of a recipient via one or more of a plurality of electrodes of a stimulating lead assembly.

31. The system of claim 27, further comprising:
means for receiving an audio signal;
means for generating a signal representative of the received audio signal; and
means for delivering the signal representative of the received audio signal to the recipient.

32. A method for providing stimulation via a stimulating lead assembly comprising a plurality of electrodes, the method comprising:
receiving a sound command specifying a sound, wherein the sound command is in compliance with a musical instrument communication protocol;
converting the sound command to at least one data signal specifying at least one stimulation signal to be provided to a cochlea of a recipient; and
providing stimulation to the cochlea in accordance with the stimulation signal.

33. The method of claim 32, wherein the sound command is a Musical Instrument Digital Interface (MIDI) command.

34. The method of claim 32, wherein providing stimulation to the cochlea comprises:
providing the stimulation signal via one or more of a plurality of electrodes of a stimulating lead assembly.

35. The method of claim 32, wherein providing stimulation to the cochlea comprises:
providing mechanical stimulation in response to the stimulation signal to at least one of an inner ear and an outer ear of the recipient.

36. A cochlear prosthesis comprising:
a sound processor configured to receive a sound command specifying a sound, wherein the sound command is in compliance with a musical instrument communication protocol, and convert the sound command to at least one data signal specifying at least one stimulation signal to be provided to a cochlea of a recipient; and
a stimulator unit configured to receive the at least one data signal, and generate the at least one stimulation signal.

37. The cochlear prosthesis of claim 36, wherein the sound command is a Musical Instrument Digital Interface (MIDI) command.

38. The cochlear prosthesis of claim 36, further comprising
a stimulating lead assembly comprising one or more electrode contacts configured to deliver the stimulation signal to the recipient's cochlea.

39. The cochlear prosthesis of claim 36, further comprising:
a stimulation arrangement configured to mechanically stimulate at least one of an inner ear or a middle ear of the recipient in response to the at least one stimulation signal.
